(19) United States
(12) Patent Application Publication
(10) Pub. No.: US 2010/0179176 A1
(43) Pub. Date: Jul. 15, 2010

(54) V3 ANTAGONISTS FOR THE TREATMENT OR PREVENTION OF CHRONIC PAIN

(75) Inventor: Inga Neumann, Regensburg (DE)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. Organon

(21) Appl. No.: 12/518,940

(22) PCT Filed: Dec. 13, 2007
(86) PCT No.: PCT/EP07/63913

§ 371 Date: Dec. 4, 2009

(51) Int. Cl. A61K 31/517; A61K 31/404; A61P 29/00; A61P 25/02; C07D 239/88; C07D 401/04; C07D 403/12

(52) U.S. Cl. 514/266.22; 514/266.31; 514/414; 544/289; 544/284; 548/465

(57) ABSTRACT

The present invention relates to a V₃ antagonist for use in the treatment of chronic pain, in particular neuropathic pain.

The present invention relates to a method of treatment of chronic pain. More particularly, the present invention relates to a vasopressin type 3 (V₃ or V_{1b}) antagonist for use in the treatment of chronic pain. The present invention further relates to a new medicinal use of V₃ antagonists.

Acute pain and chronic pain differ in their etiology, pathophysiology, diagnosis and treatment. Acute pain, which occurs following tissue injury, is self-limiting, serves as an alert to ongoing tissue damage and following tissue repair it will usually subside. There are minimal psychological symptoms associated with acute pain apart from mild anxiety. Acute pain is nociceptive in nature and occurs following chemical, mechanical and thermal stimulation of A-delta and C-polymodal pain receptors.

Chronic pain, on the other hand, serves no protective biological function. Rather than being the symptom of tissue damage it is a disease in its own right. Chronic pain is unrelenting and not self-limiting and can persist for years, perhaps decades after the initial injury. Chronic pain can be refractory to multiple treatment regimes. Psychological symptoms associated with chronic pain include chronic anxiety, fear, depression, sleeplessness and impairment of social interaction. Chronic non-malignant pain is predominantly neuropathic in nature and involves damage to either the peripheral or central nervous systems.

Acute pain and chronic pain are caused by different neuro-physiological processes and therefore tend to respond to different types of treatments. Acute pain can be somatic or visceral in nature. Somatic pain tends to be a well localised, constant pain and is described as sharp, aching, throbbing or gnawing. Visceral pain, on the other hand, tends to be vague in distribution, paroxysmal in nature and is usually described as deep, aching, squeezing or colicky in nature. Examples of acute pain include post-operative pain, pain associated with trauma and the pain of arthritis. Acute pain usually responds to treatment with opioids or non-steroidal anti-inflammatory drugs.

Chronic pain, in contrast to acute pain, is described as burning, electric, tingling and shooting in nature. It can be continuous or paroxysmal in presentation. The hallmarks of chronic pain are chronic allodynia and hyperalgesia. Allodynia is pain resulting from a stimulus that normally does not ellicit a painful response, such as a light touch.

Hyperalgesia is an increased sensitivity to normally painful stimuli. Primary hyperalgesia occurs immediately within the area of the injury. Secondary hyperalgesia occurs in the undamaged area surrounding the injury. Examples of chronic pain include complex regional pain syndromes, peripheral neuropathies, mechanical nerve injury and severe pain associated with diseases such as cancer, metabolic disease, neurotropic viral disease, neurotoxicity and multiple sclerosis. Chronic pain tends to be only partially responsive to treatment with opioid drugs.

Although opioids are cheap and effective, serious and potentially life-threatening side effects occur with their use, most notably respiratory depression and muscle rigidity. In addition the doses of opioids which can be administered are limited by nausea, emesis, constipation, pruritis and urinary retention, often resulting in patients electing to receive sub-optimal pain control rather than suffer these distressing side-effects. Furthermore, these side-effects often result in patients requiring extended hospitalisation. Opioids are highly addictive and are scheduled drugs in many territories.

Efforts continue therefore to find new treatments for pain management, in particular treatment of chronic pain. Moreover treatments of pain management are needed which are safe as well as effective.

The present invention provides a new method of treatment of chronic pain. More particularly, the present invention provides a vasopressin type 3 (V₃ or V_{1b}) antagonist for use in the treatment of chronic pain. The present invention therefore provides a method of treatment of chronic pain comprising administering a pharmaceutically effective amount of a V₃ antagonist to a subject in need thereof.

The actions of arginine vasopressin (AVP) at the pituitary corticotrope are mediated by the vasopressin type 3 (V₃ or V_{1b}) receptor, which is known and has been cloned (human receptor: Sugimoto et al., J. Biol. Chem., 1994, 269, 27088-27092). In addition to the V₃ receptor, vasopressin also activates other vasopressin receptors, i.e., the V₁ₐ receptor, found in the brain and in liver and vascular tissue and the V₂ receptor, predominantly found in kidney tissue. Interaction at these receptors mediates the pressor and antidiuretic actions of AVP.

V₃ receptor antagonists and their uses are known in the art. For example, US 2003114683 relates to certain 1,3-dihydro-2H-indol-2-one derivatives, for example SSR 149415, indicated to be useful in the treatment of CNS disorders such as stress, anxiety, depression, obsessive compulsive disorder, panic attacks, psychotic states and memory troubles. More recently WO 2006/095014 has disclosed a series of 2-(4-oxo-4H-quinazolin-3-yl)acetamide derivatives also indicated to be useful for the treatment or prevention of disorders or diseases influenced by modulation of the activity of the HPA axis, such as depression.

Database WPI, Derwent Publications Ltd., Class 065, page 1, AN 2006-5029261 XP002416515 indicates that SSR 149415 inhibits the tolerance development to the analgesic activity of morphine. There is no suggestion, however, of the possibility that SSR 149415 would be effective, in its own right, in the treatment of chronic pain. Furthermore, there is no suggestion that SSR 149415 would be effective in the treatment of chronic pain whilst showing little or no effect in the treatment of acute pain as has now been found. US 2003114683 indicates that the V₃ antagonists disclosed therein are useful in a range of disorders including the treatment of pain in general and pain-perception disorders although no supporting experimental data is provided. However likewise, there is no suggestion in US 2003114683 of the possibility that V₃ antagonists might be especially effective in the treatment of chronic pain whilst showing little or no effect against acute pain as has now been found.

In one embodiment of the present invention is a V₃ receptor antagonist having the formula I:

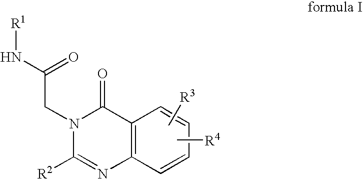

wherein
R¹ is C₁₋₆alkyl, C₃₋₆cycloalkyl, C₃₋₆cycloalkylC₁₋₂alkyl, C₂₋₆alkenyl or C₂₋₆alkynyl, said C₁₋₆alkyl, C₃₋₆cycloalkyl and C₃₋₆cycloalkylC₁₋₂alkyl being optionally substituted with one or more halogens;
R² is C₆₋₁₀aryl optionally substituted with one to three substituents selected from halogen, hydroxy, cyano, C₁₋₆alkyl, C₃₋₆cycloalkyl, C₁₋₆alkyloxy and C₃₋₆cycloalkyloxy, said C₁₋₆alkyl, C₃₋₆ cycloalkyl, C₁₋₆ alkyloxy and C₃₋₆cycloalkyloxy being optionally substituted with one or more halogens or R² is a 5-10 membered heteroaryl ring system comprising a heteroatom selected from N, O, S and optionally substituted with a substituent selected from methyl, C₁₋₆alkyloxy and halogen;
R³ is an optional substituent selected from C₁₋₆alkyl, C₁₋₆alkyloxy and halogen, said C₁₋₆alky and C₁₋₆alkyloxy being optionally substituted with one or more halogens;
R⁴ is a group located at the 6- or 7-position of the quinazolinone ring and is selected from

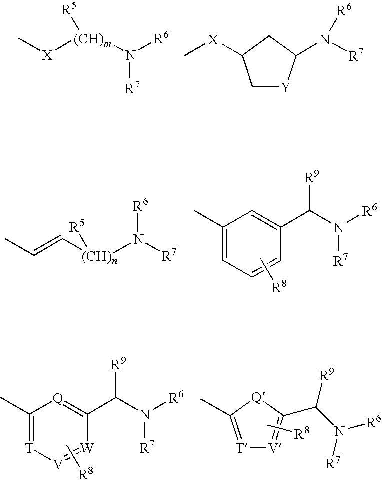

each R⁵ is independently H or C₁₋₆ alkyl or one of R⁵ when joined together with one of R⁶ or R⁷ forms a 4-7 membered heterocyclic ring;
R⁶ and R⁷ are independently H, C₁₋₆alkyl, C₃₋₆cycloalkyl, C₃₋₆cycloalkylC₁₋₂alkyl, C₆₋₁₀ aryl or C₆₋₁₀arylC₁₋₂alkyl; or R⁶ and R⁷ together with the nitrogen to which they are bonded form a 4 to 8 membered saturated or unsaturated heterocyclic ring optionally comprising a further heteroatomic moiety selected from O, S and NR¹⁰, said heterocyclic ring being optionally substituted with one or two substituents selected from halogen, hydroxyl, C₁₋₆alkyl, C₁₋₆alkyloxy, cyano and COOR¹¹ and said heterocyclic ring being optionally fused at two adjacent carbon atoms to a phenyl ring;
   or one of R⁶ and R⁷ when joined together with one of R⁵ forms a 4-7 membered heterocyclic ring;
   or one of R⁶ and R⁷ when joined together with one of R⁸ forms a 5-6 membered heterocyclic ring;
R⁸ is one or two substituents selected from H, C₁₋₆alkyl, C₁₋₆alkyloxy and halogen or one of R⁸ when joined together with one of R⁶ and R⁷ forms a 5-6 membered heterocyclic ring;
   or one of R⁸ when joined together with R⁹ forms a 5-6 membered ring
R⁹ is H or C₁₋₆alkyl or R⁹ when joined together with one of R⁸ forms a 5-6 membered ring;
R¹⁰ is H, C₁₋₆alkyl or C₁₋₆acyl;
R¹¹ is H or C₁₋₆alkyl;
m is 2-4;
n is 1-3;
X is CH₂, O, S, SO₂ or NR¹²;
R¹² is H, C₁₋₆alkyl, C₁₋₆acyl or C₆₋₁₀arylC₁₋₂alkyl group, said C₆₋₁₀arylC₁₋₂alkyl group being optionally substituted with methyl or methoxy;
Y is CH₂, (CH₂)₂ or (CH₂)₃;
Q, T, V and W are C or N with the proviso that one of Q, T, V and W is N and the others are C;
Q′, T′ and V′ are selected from C, O, N and S with the proviso that one of Q′, T′ and V′ is O, N, or S and the others are C;
   or a pharmaceutically acceptable salt or solvate thereof for the manufacture of a medicament for the treatment or prevention of chronic pain.

The term C₁₋₆alkyl, as used herein, represents a branched or unbranched alkyl group having 1-6 carbon atoms. Examples of such groups are methyl, ethyl, isopropyl, tertiary-butyl, pentyl and hexyl.

The term C₂₋₆alkenyl, as used herein, represents a branched or unbranched alkenyl group having 2-6 carbon atoms and at least one double bond. Examples of such groups are ethenyl and 3-methylbutynyl.

The term C₂₋₆alkynyl, as used herein, represents a branched or unbranched alkynyl group having 2-6 carbon atoms and at least one triple bond. Examples of such groups are ethynyl and 3-methylbutynyl.

The term C₃₋₆cycloalkyl, as used herein, represents a branched or unbranched cyclic alkyl group having 3-6 carbon atoms. Examples of such groups are cyclopropyl, cyclopentyl and 2-methylcyclopentyl.

The term C₃₋₆cycloalkylC₁₋₂alkyl, as used herein, represents a C₁₋₂ alkyl group which is substituted with a C₃₋₆cycloalkyl group. Examples of such groups are cyclopropylmethyl and 2-cyclobutylethyl.

The term C₁₋₆alkyloxy, as used herein, represents a branched or unbranched alkyloxy group having 1-6 carbon atoms. Examples of such groups are methoxy, ethoxy, isopropyloxy and tertiary-butyloxy.

The term C₃₋₆cycloalkyloxy, as used herein, represents a branched or unbranched cyclic alkyloxy group having 3-6 carbon atoms. Examples of such groups are cyclopropyloxy, cyclopentyloxy and 2-methylcyclopentyloxy. Similarly, the term C₄₋₆ cycloalkyloxy represents a branched or unbranched cyclic alkyloxy group having 4-6 carbon atoms.

The term C₁₋₆acyl, as used herein, represents an acyl group derived from a carboxylic acid having 1-6 carbon atoms. The acyl group can comprise a hydrocarbon which may be branched, unbranched, saturated or unsaturated. Examples of such groups include formyl, acetyl, propionyl, acryloyl and pivaloyl. Also included within the definition of C₁₋₆ acyl are groups derived from dicarboxylic acids like groups derived from malonic acid.

The term C₆₋₁₀aryl, as used herein, represents an aromatic group having 6-10 carbon atoms. Examples of such groups include phenyl and naphthyl.

The term C₆₋₁₀arylC₁₋₂alkyl, as used herein, represents a C₁₋₂alkyl group which is substituted with a C₆₋₁₀aryl group. Examples of such groups include benzyl and phenethyl.

The term halogen, as used herein, represents a fluorine, chlorine, bromine or iodine.

The term 5-10 membered heteroaryl ring system comprising a heteroatom selected from N, O and S, as used herein, represents a monocyclic or fused bicyclic 5-10 membered heteroaryl ring system comprising a heteroatom selected from N, O and S. Examples of such groups include furanyl, thienyl, pyrrolyl, pyridinyl, indolyl, benzthienyl and quinolinyl.

Examples of 4 to 8 membered saturated or unsaturated heterocyclic rings formed by R⁶ and R⁷ together with the nitrogen to which they are bonded and optionally comprising a further heteroatomic moiety selected from O, S and NR¹⁰ wherein R⁶, R⁷ and R¹⁰ have the previously defined meanings, as used herein, include piperidine, homopiperidine, morpholine, thiomorpholine, 4-methylpiperazine, tetrahydropyridine and 4-methylhomopiperazine.

Methods for the preparation of the V₃ antagonists of this embodiment of the present invention having the formula I are described in WO 2006/095014.

The present invention also includes within its scope all stereoisomeric forms of the V₃ antagonists as described herein resulting, for example, because of configurational or geometrical isomerism. Such stereoisomeric forms are enantiomers, diastereoisomers, cis and trans isomers etc. For example, in the case where R¹ is 2-methylbutyl the compound exists as a pair of enantiomers. In the case where R⁴ comprises an alkene fragment, both (Z) and (E) stereoisomeric forms of the compound are possible. In the case of the individual enantiomers of compounds of formula I or salts or solvates thereof, the present invention includes use of the aforementioned stereoisomers substantially free, i.e., associated with less than 5%, preferably less than 2% and in particular less than 1% of the other enantiomer. Mixtures of stereoisomers in any proportion, for example a racemic mixture comprising substantially equal amounts of two enantiomers are also included within the scope of the present invention.

In a further embodiment of the present invention is a V₃ receptor antagonist for use in the treatment of chronic pain, wherein said V₃ receptor antagonist is selected from:
(2S,4R)-1-[5-chloro-1-(2,4-dimethoxyphenyl)sulfonyl]-3-(2-methoxyphenyl)-2-oxo-2,3-dihydro-1H-indol-3-yl]-4-hydroxy-N,N-dimethyl-2-pyrrolidine carboxamide;
2-[2-(3-Chloro-4-fluorophenyl)-4-oxo-6-(3-piperidin-1-ylpropoxy)-4H-quinazolin-3-yl]-N-isopropylacetamide;
N-Isopropyl-2-[2-(3-methoxyphenyl)-4-oxo-6-(3-piperidin-1-ylpropoxy)-4H-quinazolin-3-yl]acetamide;
2-[2-(4-Fluoro-3-methoxyphenyl)-4-oxo-6-(3-piperidin-1-ylpropoxy)-4H-quinazolin-3-yl]-N-isopropylacetamide;
2-[2-(3-Chlorophenyl)-6-[3-(4-hydroxypiperidin-1-yl)propoxy]-4-oxo-4H-quinazolin-3-yl]-N-isopropylacetamide;
2-[2-(3-Chlorophenyl)-4-oxo-6-(3-piperidin-1-ylpropoxy)-4H-quinazolin-3-yl]-N-isopropylacetamide;
(S)-(+)-2-[2-(3-Chlorophenyl)-6-(2-methyl-3-pyrrolidin-1-ylpropoxy)-4-oxo-4H-quinazolin-3-yl]-N-isopropylacetamide;
2-[6-(5-Dimethylaminomethyl-2-fluorophenyl)-2-(3-methoxyphenyl)-4-oxo-4H-quinazolin-3-yl]-N-isopropylacetamide;
2-[6-(3-Dimethylaminomethylphenyl)-2-(3-methoxyphenyl)-4-oxo-4H-quinazolin-3-yl]-N-isopropylacetamide;
N-tert-Butyl-2-[2-(3-chlorophenyl)-4-oxo-6-(3-pyrrolidin-1-ylpropoxy)-4H-quinazolin-3-yl]acetamide and
2-[6-(3-Dimethylaminomethylphenyl)-2-(4-fluoro-3-methoxyphenyl)-4-oxo-4H-quinazolin-3-yl]-N-isopropylacetamide
   or a pharmaceutically acceptable salt or solvate thereof.

In a further embodiment is a V₃ antagonist for use in the treatment of chronic pain, wherein said chronic pain is a complex regional pain syndrome, pain arising from peripheral neuropathies, post-operative pain, chronic fatigue syndrome pain, tension-type headache, pain arising from mechanical nerve injury and severe pain associated with diseases such as cancer, metabolic disease, neurotropic viral disease, neurotoxicity, inflammation, multiple sclerosis or any pain arising as a consequence of or associated with stress or depressive illness. In a further embodiment is the use of a V₃ antagonist for the manufacture of a medicament for the treatment or prevention of chronic pain wherein said chronic pain is neuropathic pain.

Pharmaceutical compositions for the use as claimed and described herein were prepared in accordance with standard techniques in the art of pharmaceutical sciences. The compounds can be used for humans in a dosage of 0.001-50 mg per kg body weight, preferably in a dosage of 0.01-20 mg per kg body weight, whereby the optimum dosage can be determined according to factors such as route of administration, desired duration of action, type of formulation (extended release versus immediate release), type of patient, type of compound required, efficacy of the compound and other physical characteristics of the recipient of the treatment, such co-morbidity of other diseases, liver metabolism capacity, etc.

V₃ receptor antagonism and methods how to determine such a biological effect can be determined according to known techniques in the biochemistry of G-protein coupled receptors. A specific method is described in the example below, on which basis a criterion pKi value of at least 6.0, or preferably 6.5, or even better 7.0 can be derived for clarity of the meaning of the term V₃ receptor antagonist. Further V₃ receptor antagonists can be identified in this way.

For diagnostic criteria for chronic pain reference is made to the DSM IV revised edition.

The invention is further illustrated by the following examples which are not intended to limit the scope thereof:

### EXAMPLE 1

### Determination of V₃ receptor Antagonism

Chinese Hamster Ovary (CHO) cells stably expressing the human V₃ receptor were incubated to equilibrium with the test compound (at a final assay concentration of 10⁻¹⁰ mol·L⁻¹ to 10⁻⁵ mol·L⁻¹) and [³H]AVP (at a final assay concentration of 2.5×10⁻⁹ mol·L⁻¹). Throughout the concentration of dimethylsulphoxide (DMSO) did not exceed 0.1% (v/v). After washing with ice-cold phosphate buffered saline (PBS), scintillation fluid was added and the plates counted on a Topcount NXT apparatus.

A sigmoidal dose response curve (non-linear regression, variable slope) was plotted as concentration of test compound (mol·L⁻¹) against percentage specific binding of [³H]AVP and a Kᵢ value was calculated. Each determination was carried out in triplicate and repeated on at least 3 separate occasions

The ability of compounds of the invention to act as V₃ antagonists in a physiologically relevant system was determined by measuring their ability to block the release of adrenocorticotropic hormone (ACTH) from anterior pituitary corticotrophs in response to treatment with arginine vasopressin (AVP).

Anterior pituitary corticotrophs were prepared from adult female Sprague-Dawley rats and seeded into 48 well plates. The cells were cultured for 4 days prior to exposure to compound. Test compounds were prepared at 10⁻⁵ mol·L⁻¹ in 100% DMSO. Cells were exposed to a dose response of test compounds for 20 minutes (10⁻⁸ mol·L⁻¹-10⁻⁵ mol·L⁻¹). The final concentration of DMSO in the assay was kept constant at 0.3%. The cells were then exposed to 3×10⁻⁹ AVP for 120 minutes. Supernatants were harvested and stored at −20° C. ACTH levels were subsequently measured by ELISA following the manufacturer's instructions (Immunodiagnostic systems, UK (Cat No. DX-SDX018)). Each treatment was carried out in quadruplicate and a mean value obtained for the amount of ACTH released. The degree of antagonism was then calculated as a percentage of the amount of ACTH released by agonist alone after adjustment for basal levels of ACTH. A pIC₅₀ was calculated by fitting a Sigmoidal dose response (variable slope) curve with a non-linear (fit) to the data using the software package GraphPad prism. Each determination was repeated on at least 3 separate occasions

### EXAMPLE 2

### Evaluation of Selective V₃ Receptor Antagonists in a Rat (Chung) Model of Neuropathic Pain

In this model, mechanical allodynia is induced by tight ligation of the left L5 spinal nerve. This assay has been employed successfully to demonstrate anti-allodynic effects of anticonvulsants (gabapentin), antidepressants (duloxetine) and opioid analgesics (morphine) which are used clinically in the treatment of neuropathic pain.

Male Wistar rats (150-175 g body weight at time of surgery) were employed in the study. Rats were placed on an elevated (˜40 cm) mesh floor in perspex boxes and the rats' withdrawal threshold to a mechanical stimulus (calibrated von Frey filaments) was measured using filaments of increasing force (2.6-167 mN). The von Frey filaments were applied to the plantar surface of the paw and the thershold response was determined using the up and down method. A positive response was noted if the paw was sharply withdrawn. A cut-off of 15 g was selected as the upper limit for testing. Following baseline measurements each animal was anaesthetised and the L5 spinal nerve tightly ligated. The animals were allowed to recover from the surgery for a period of at least three days. On the day of drug administration the paw withdrawal thresholds were re-measured (0 min). Immediately after this reading, the rats were dosed orally with vehicle or test compound. Readings were then made at intervals of 30, 60, 120, 180 and 240 min after compound administration.

Data were expressed as mean±s.e.m. The time of maximum effect for each animal in the top dose group was determined and these values averaged to calculate the mean time of maximum effect. For analytical purposes the time of maximum effect, tₘₐₓ was defined as the time point closest to this averaged value. Data at tₘₐₓ were compared between groups using the Kruskal-Wallis one-way analysis of variance, a non-parametric statistical test. Each of the treatment groups were then compared against the vehicle group, using the non-parametric Dunn's test. The ED₅₀ (dose at which allodynia is reversed by 50%) value was also calculated at tₘₐₓ using non linear regression (sigmoidal dose response; variable slope) with constants of 0 and 15 g (cut-off) for the bottom and top, respectively (XLFit software).

Three V₃ antagonists were used in the experiment:
Compound 1, i.e., 2-[6-(3-Dimethylaminomethylphenyl)-2-(4-fluoro-3-methoxyphenyl)-4-oxo-4H-quinazolin-3-yl]-N-isopropylacetamide;
Compound 2, i.e., 2-[3-(3-chlorophenyl)-4-oxo-6-(3-piperidin-1-ylpropoxy)-4H-quinazolin-3-yl]-N-isopropylacetamide and
Compound 3, i.e., (2S,4R)-1-[5-chloro-1-(2,4-dimethoxyphenyl)sulfonyl]-3-(2-methoxyphenyl)-2-oxo-2,3-dihydro-1H-indol-3-yl]-4-hydroxy-N,N-dimethyl-2-pyrrolidine carboxamide i.e., SSR149415;

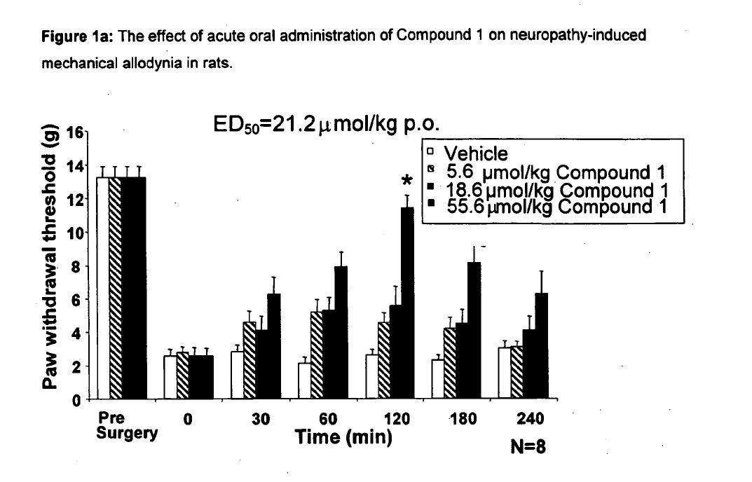

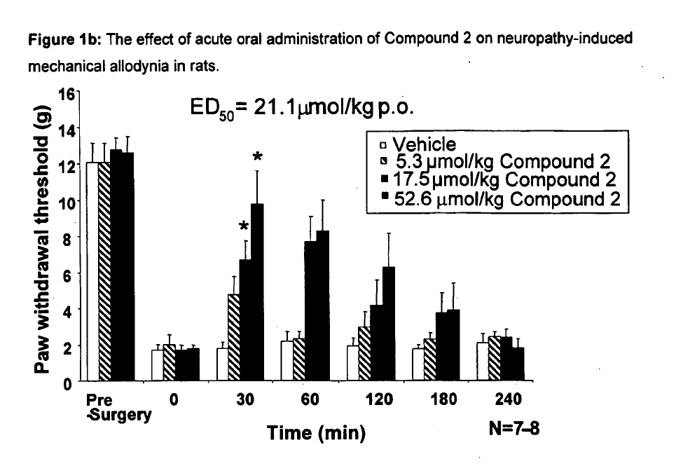

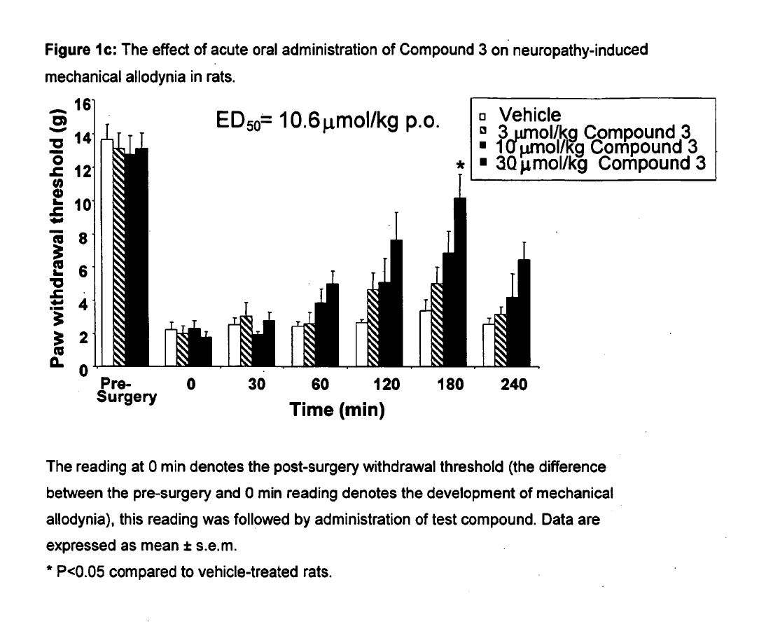

Oral administration of these compounds, compound 1, (5.6, 18.6 or 55.6 μmol/kg), compound 2, (5.3, 17.3 or 52.6 μmol/kg) or compound 3, (3, 10, or 30 μmol/kg) in rats reversed mechanical allodynia in a dose-dependent fashion (Table 1; FIGS. 1a, b, c, respectively). The peak anti-allodynic effect for compound 1 was observed at 120 min (FIG. 1a), for compound 2 at 30 min (FIG. 1b) and for compound 3 at 180 min (FIG. 1c) post drug administration. The calculated ED₅₀ values at peak effect were 21.2 μmol/kg p.o. for compound 1, 21.0 μmol/kg for compound 2 and 10.6 μmol/kg for compound 3.

These data demonstrate that V₃ receptor antagonists possess potent oral anti-allodynic activity in a rat model of neuropathic pain, a form of chronic pain.

TABLE 1
Effect of 3 V₃ antagonists on mechanical allodynia induced by spinal
nerve ligation in rats. Dose groups and number of animals per group.

| | | | | Number of | Withdrawal |
|---|---|---|---|---|---|
| | | Dose | | animals | threshold (g) at |
| Compound | Route | (μmol/kg) | | tested | peak effect |
| Vehicle | p.o. | 1 | ml · kg⁻¹ | 8 | 2.6 ± 0.3 |
| Compound 1 | p.o. | 5.6 | | 8 | 4.6 ± 0.6 |
| Compound 1 | p.o. | 18.6 | | 8 | 5.6 ± 1.1 |
| Compound 1 | p.o. | 55.6 | | 8 | 11.4 ± 0.7** |
| Vehicle | p.o. | 1 | ml · kg⁻¹ | 8 | 1.8 ± 0.3 |
| Compound 2 | p.o. | 5.3 | | 8 | 4.8 ± 1.0 |
| Compound 2 | p.o. | 17.3 | | 8 | 6.7 ± 1.1* |
| Compound 2 | p.o. | 52.6 | | 7 | 9.8 ± 1.8** |
| Vehicle | p.o. | 1 | ml · kg−1 | 7 | 3.4 ± 0.7 |
| Compound 3 | p.o. | 3 | | 7 | 5.0 ± 1.0 |
| Compound 3 | p.o. | 10 | | 7 | 6.9 ± 1.3 |
| Compound 3 | p.o. | 30 | | 7 | 10.1 ± 1.4** |

*p ≦ 0.01
**p ≦ 0.001,
Dunns test comparing vehicle-treated and compound-treated animals.

### EXAMPLE 3

### Evaluation of the V₃ Receptor Antagonist Compound 2 on Mechanical Hyperalgesia in a Rat Model of Inflammatory Pain

In this model, inflammation is induced by subcutaneous injection of complete Freund's adjuvant (CFA) into the hind paw. The associated mechanical hyperalgesia is quantified by measuring the reduction in paw withdrawal threshold (PWT) to a mechanical compression of the paw. This assay has been employed successfully to demonstrate anti-hyperalgesic effects of non-steroidal anti-inflammatory drugs (indomethacin) and cyclooxygenase-2 inhibitors (celecoxib) which are used clinically in the treatment of chronic inflammatory pain.

Experiments were in male Wistar rats weighing (130-180 g). In brief, the rats' paw withdrawal threshold (PWT) to a mechanical compression of the hind paw was measured (baseline reading) using a Randall-Sellito apparatus (Ugo Basile). A cut-off of 200 g was employed to minimise tissue damage to the paw. The animals were then lightly anaesthetised with isoflurane (1-3%) and CFA (0.1 ml·paw⁻¹) injected subcutaneously (s.c.) into the plantar surface of the left hind paw. The animals were then returned to their home cage and left for the inflammation to develop.

Seven days after CFA injection, PWT's were re-measured (0 min) and immediately after this reading, rats were dosed orally with either vehicle or compound 2 (3-30 mg/kg). Readings were then made at 30 min and 2 h post drug administration.

Data were plotted as mean±s.e.m. and compared between groups using the Kruskal-Wallis one-way analysis of variance, a non-parametric statistical test. If statistical significance (P<0.05) was observed with this test, the vehicle group and each of the treatment groups were compared using the non-parametric Dunn's test. The percent attenuation of mechanical hyperalgesia is calculated at time of peak effect (tₘₐₓ)

$$\% attenuation of hyperalgesia = \frac{( Post compound PWT - post CFA PWT )}{( Baseline PWT - post CFA PWT )} \times 100$$

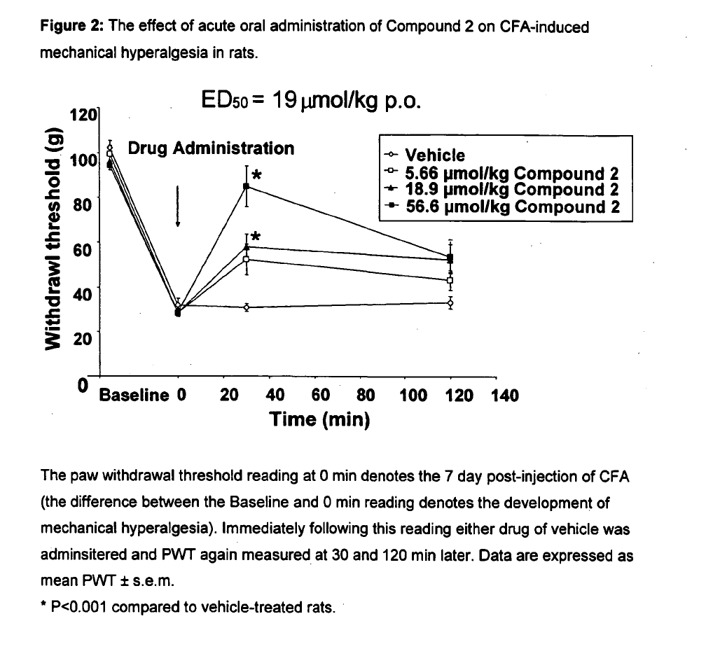

Oral administration of Compound 2 (5.7, 18.9 or 56.6 μmol/kg) reversed mechanical hyperalgesia induced by CFA in a dose-dependent fashion (Table 2; FIG. 2). The peak anti-algesic effect was observed at 30 min post drug administration (FIG. 2). The calculated ED₅₀ value at peak effect was 19.2 μmol/kg p.o.

These data demonstrate that the V₃ receptor antagonist Compound 2 possesses potent oral anti-algesic activity in a rat model of chronic inflammatory pain.

TABLE 2
Effect of Compound 2 on mechanical hyperalgesia induced by
complete Freund's adjuvant administered 7 days previously
in rats. Dose groups and number of animals per group.

| | | | | Number of | % Attenuation |
|---|---|---|---|---|---|
| | | Dose | | animals | of hyperalgesia |
| Compound | Route | (μmol/kg) | | tested | (peak effect) |
| Vehicle | p.o. | 3 | ml · kg⁻¹ | 8 | −2.2 ± 2.6 |
| Compound 2 | p.o. | 5.7 | | 8 | 31.5 ± 9.4 |
| Compound 2 | p.o. | 18.9 | | 8 | 43.1 ± 8.6** |
| Compound 2 | p.o. | 56.6 | | 8 | 84.4 ± 12.0** |

*p ≦ 0.01
**p ≦ 0.001,
Dunns test comparing vehicle-treated and compound-treated animals.

### EXAMPLE 4

### Evaluation of the V₃ Receptor Antagonists in Acute Thermal Pain Using the Mouse Tail Flick Assay

The tail flick test is used to evaluate the antinociceptive activity of test compounds in mice. Animals are placed on the tail flick apparatus and their tails exposed to a focused beam of radiant heat. The mice react to the noxious thermal stimulus by flicking their tail away from the heat source. The time taken for these responses to occur is measured (latency; s). This assay has been employed successfully to demonstrate antinociceptive effects of opioids (morphine, fentanyl) and adrenergic agonists (clonidine) which are used clinically for the treatment of moderate pain.

Experiments were performed using male ICR mice weighing (25-32 g). In brief, animals were held in place on the tail flick apparatus (Ugo Basile, Italy) by means of a perspex restrainer and their tail was positioned over the heat source. The latency to flick the tail from the radiant heat source focussed 2.5 cm from the tip was determined before (baseline readings) and at regular intervals after administration of the vehicle or test compounds (compound 1, 5.6-55.6 μmol/kg p.o.; compound 2, 5.3-52.6 μmol/kg p.o.). A 12 seconds cut-off was employed to prevent tissue damage.

Data are expressed as mean±s.e.m. and compared between groups using the Kruskal-Wallis one-way analysis of variance, a non-parametric statistical test. If statistical significance (P<0.05) is observed with this test, the vehicle group and each of the treatment groups is compared using the non-parametric Dunn's test.

Oral administration of compound 1, (5.6, 18.6 or 55.6 μmol/kg) or compound 2, (5.3, 17.3 or 52.6 μmol/kg) to mice had no effect on tail flick latencies when measured 20, 40 and 60 min post compound administration (Table 3).

These data demonstrate that the V₃ receptor antagonists do not posses antinociceptive activity in a mouse model of acute thermal pain.

TABLE 3
Lack of effect of Compound 1 and 2 on tail flick latency in mice.

| | | | Number of | Tail Flick Latency (s) | | |
|---|---|---|---|---|---|---|
| | | Dose | animals | 20 min | 40 min | 60 min |
| Compound | Route | (μmol/kg) | tested | post dose | post dose | post dose |
| Vehicle | p.o. | 1 ml · kg⁻¹ | 8 | 3.4 ± 0.3 | 3.2 ± 0.2 | 3.3 ± 0.1 |
| Compound 1 | p.o. | 5.6 | 8 | 3.5 ± 0.4 | 3.4 ± 0.2 | 3.5 ± 0.2 |
| Compound 1 | p.o. | 18.6 | 8 | 3.4 ± 0.3 | 3.5 ± 0.2 | 3.1 ± 0.2 |
| Compound 1 | p.o. | 55.6 | 8 | 3.3 ± 0.3 | 3.5 ± 0.3 | 3.3 ± 0.2 |
| Vehicle | p.o. | 1 ml · kg⁻¹ | 8 | 3.2 ± 0.1 | 3.3 ± 0.1 | 3.3 ± 0.1 |
| Compound 2 | p.o. | 5.3 | 8 | 3.4 ± 0.2 | 3.7 ± 0.2 | 3.6 ± 0.1 |
| Compound 2 | p.o. | 17.3 | 8 | 3.6 ± 0.1 | 3.6 ± 0.1 | 3.1 ± 0.1 |
| Compound 2 | p.o. | 52.6 | 8 | 3.5 ± 0.2 | 3.5 ± 0.2 | 3.2 ± 0.2 |

Dose groups and number of animals per group. No significant difference was observed using Kruskal-Wallis analysis of variance at any of the time points.

1-3. (canceled)

4. A method of treatment of chronic pain comprising administering a pharmaceutically effective amount of a V₃ antagonist to a subject in need thereof.

5. The method of treatment according to claim 4, wherein the V₃ antagonist is selected from:
(2S,4R)-1-[5-chloro-1-(2,4-dimethoxyphenyl)sulfonyl]-3-(2-methoxyphenyl)-2-oxo-2,3-dihydro-1H-indol-3-yl]-4-hydroxy-N,N-dimethyl-2-pyrrolidine carboxamide;
2-[2-(3-Chloro-4-fluorophenyl)-4-oxo-6-(3-piperidin-1-ylpropoxy)-4H-quinazolin-3-yl]-N-isopropylacetamide;
N-Isopropyl-2-[2-(3-methoxyphenyl)-4-oxo-6-(3-piperidin-1-ylpropoxy)-4H-quinazolin-3-yl]acetamide;
2-[2-(4-Fluoro-3-methoxyphenyl)-4-oxo-6-(3-piperidin-1-ylpropoxy)-4H-quinazolin-3-yl]-N-isopropylacetamide;
2-[2-(3-Chlorophenyl)-6-[3-(4-hydroxypiperidin-1-yl)propoxy]-4-oxo-4H-quinazolin-3-yl]-N-isopropylacetamide;
2-[2-(3-Chlorophenyl)-4-oxo-6-(3-piperidin-1-ylpropoxy)-4H-quinazolin-3-yl]-N-isopropylacetamide;
(S)-(+)-2-[2-(3-Chlorophenyl)-6-(2-methyl-3-pyrrolidin-1-ylpropoxy)-4-oxo-4H-quinazolin-3-yl]-N-isopropylacetamide;
2-[6-(5-Dimethylaminomethyl-2-fluorophenyl)-2-(3-methoxyphenyl)-4-oxo-4H-quinazolin-3-yl]-N-isopropylacetamide;
2-[6-(3-Dimethylaminomethylphenyl)-2-(4, fluoro-3-methoxyphenyl)-4-oxo-4H-quinazolin-3-yl]-N-isopropylacetamide;
N-tert-Butyl-2-[2-(3-chlorophenyl)-4-oxo-6-(3-pyrrolidin-1-ylpropoxy)-4H-quinazolin-3-yl]acetamide and
2-[6-(3-Dimethylaminomethylphenyl)-2-(3-methoxyphenyl)-4-oxo-4H-quinazolin-3-yl]-N-isopropylacetamide
or a pharmaceutically acceptable salt or solvate thereof.

6. The method of treatment according to claim 4, wherein the chronic pain is neuropathic pain.

7. The method of treatment according to claim 4, wherein the chronic pain is inflammatory pain.

8. The method of treatment according to claim 4, wherein the subject is a mammal.

9. The method of treatment according to claim 8, wherein the mammal is human.

10. A pharmaceutical composition comprising a V₃ antagonist selected from (2S,4R)-1-[5-chloro-1-(2,4-dimethoxyphenyl)sulfonyl]-3-(2-methoxyphenyl)-2-oxo-2,3-dihydro-1H-indol-3-yl]-4-hydroxy-N,N-dimethyl-2-pyrrolidine carboxamide;
2-[2-(3-Chloro-4-fluorophenyl)-4-oxo-6-(3-piperidin-1-ylpropoxy)-4H-quinazolin-3-yl]-N-isopropylacetamide;
N-Isopropyl-2-[2-(3-methoxyphenyl)-4-oxo-6-(3-piperidin-1-ylpropoxy)-4H-quinazolin-3-yl]acetamide;
2-[2-(4-Fluoro-3-methoxyphenyl)-4-oxo-6-(3-piperidin-1-ylpropoxy)-4H-quinazolin-3-yl]-N-isopropylacetamide;
2-[2-(3-Chlorophenyl)-6-[3-(4-hydroxypiperidin-1-yl)propoxy]-4-oxo-4H-quinazolin-3-yl]-N-isopropylacetamide;
2-[2-(3-Chlorophenyl)-4-oxo-6-(3-piperidin-1-ylpropoxy)-4H-quinazolin-3-yl]-N-isopropylacetamide;
(S)-(+)-2-[2-(3-Chlorophenyl)-6-(2-methyl-3-pyrrolidin-1-ylpropoxy)-4-oxo-4H-quinazolin-3-yl]-N-isopropylacetamide;
2-[6-(5-Dimethylaminomethyl-2-fluorophenyl)-2-(3-methoxyphenyl)-4-oxo-4H-quinazolin-3-yl]-N-isopropylacetamide;
2-[6-(3-Dimethylaminomethylphenyl)-2-(4, fluoro-3-methoxyphenyl)-4-oxo-4H-quinazolin-3-yl]-N-isopropylacetamide;
N-tert-Butyl-2-[2-(3-chlorophenyl)-4-oxo-6-(3-pyrrolidin-1-ylpropoxy)-4H-quinazolin-3-yl]acetamide and
2-[6-(3-Dimethylaminomethylphenyl)-2-(3-methoxyphenyl)-4-oxo-4H-quinazolin-3-yl]-N-isopropylacetamide
or a pharmaceutically acceptable salt or solvate thereof.
